SEQUENCE LISTING (103 sequences)

SEQ ID NO: 1 - DNA, 22 bp, Artificial Sequence
  Oligonucleotide, Accession number D01150, Forward Primer (5`-3`)
tcacagacacagcctgattgag

SEQ ID NO: 2 - DNA, 20 bp, Artificial Sequence
  Oligonucleotide, Accession number D01150, Reverse Primer (5`-3`)
gatgggttgacccatagctt

SEQ ID NO: 3 - DNA, 23 bp, Artificial Sequence
  Oligonucleotide, Accession number M38504, Forward Primer (5`-3`)
tggcttctacatccccaagaaat

SEQ ID NO: 4 - DNA, 20 bp, Artificial Sequence
  Oligonucleotide, Accession number M38504, Reverse Primer (5`-3`)
ttcatggtcagcccgtagat

SEQ ID NO: 5 - DNA, 18 bp, Artificial Sequence
  Oligonucleotide, Accession number U22027, Forward Primer (5`-3`)
gtgtggacatgatgccgt

SEQ ID NO: 6 - DNA, 18 bp, Artificial Sequence
  Oligonucleotide, Accession number U22027, Reverse Primer (5`-3`)
aggacttgaggcggaagt

SEQ ID NO: 7 - DNA, 20 bp, Artificial Sequence
  Oligonucleotide, Accession number X13494, Forward Primer (5`-3`)
ccatacacagaggcagtcat

SEQ ID NO: 8 - DNA, 20 bp, Artificial Sequence
  Oligonucleotide, Accession number X13494, Reverse Primer (5`-3`)
ggtgtcagatcgatgtcttc

SEQ ID NO: 9 - DNA, 22 bp, Artificial Sequence
  Oligonucleotide, Accession number XM_050922, Forward Primer (5`- 3`)
gatcatgtaattggcagacaca

SEQ ID NO: 10 - DNA, 21 bp, Artificial Sequence
  Oligonucleotide, Accession number XM_050922, Reverse Primer (5`- 3`)
cctgctgagaaaggcatgaag

SEQ ID NO: 11 - DNA, 20 bp, Artificial Sequence
  Oligonucleotide, Accession number XM_050918, Forward Primer (5`- 3`)
agcttggaaaacactgcagt

SEQ ID NO: 12 - DNA, 21 bp, Artificial Sequence
  Oligonucleotide, Accession number XM_050918, Reverse Primer (5`- 3`)
cctgctgagaaaggcatgaag

SEQ ID NO: 13 - DNA, 20 bp, Artificial Sequence
  Oligonucleotide, Accession number NM_000772, Forward Primer (5`- 3`)
ctgtaactgatatgtttggg

SEQ ID NO: 14 - DNA, 21 bp, Artificial Sequence
  Oligonucleotide, Accession number NM_000772, Reverse Primer (5`- 3`)
cctgctgagaaaggcatgaag

SEQ ID NO: 15 - DNA, 22 bp, Artificial Sequence
  Oligonucleotide, Accession number NM_000769, Forward Primer (5`- 3`)
gtaatcactgcagctgacttac

SEQ ID NO: 16 - DNA, 21 bp, Artificial Sequence
  Oligonucleotide, Accession number NM_000769, Reverse Primer (5`- 3`)
cctgctgagaaaggcatgaag

SEQ ID NO: 17 - DNA, 20 bp, Artificial Sequence
  Oligonucleotide, Accession number M33189, Forward Primer (5`-3`)
tgatgagaacctgcgcatag

SEQ ID NO: 18 - DNA, 20 bp, Artificial Sequence
  Oligonucleotide, Accession number M33189, Reverse Primer (5`-3`)
accgatgacaggttggtgat

SEQ ID NO: 19 - DNA, 20 bp, Artificial Sequence
  Oligonucleotide, Accession number AF084225, Forward Primer (5`-3`)
agcacaactctgagatatgg

SEQ ID NO: 20 - DNA, 20 bp, Artificial Sequence
  Oligonucleotide, Accession number AF084225, Reverse Primer (5`-3`)
atagtcactgtacttgaact

SEQ ID NO: 21 - DNA, 20 bp, Artificial Sequence
  Oligonucleotide, Accession number J02906, Forward Primer (5`-3`)
atgaacttgccgcaccgcgt

SEQ ID NO: 22 - DNA, 20 bp, Artificial Sequence
  Oligonucleotide, Accession number J02906, Reverse Primer (5`-3`)
acaggctccacttacggtgc

SEQ ID NO: 23 - DNA, 20 bp, Artificial Sequence
  Oligonucleotide, Accession number X12387, Forward Primer (5`-3`)
ccaagctatgctcttcaccg

SEQ ID NO: 24 - DNA, 20 bp, Artificial Sequence
  Oligonucleotide, Accession number X12387, Reverse Primer (5`-3`)
tcaggctccacttacggtgc

SEQ ID NO: 25 - DNA, 20 bp, Artificial Sequence
  Oligonucleotide, Accession number L26985, Forward Primer (5`-3`)
tgtccagcagaaactgcaaa

SEQ ID NO: 26 - DNA, 21 bp, Artificial Sequence
  Oligonucleotide, Accession number L26985, Reverse Primer (5`-3`)
ttgaagaagtccttgcgtgtc

SEQ ID NO: 27 - DNA, 20 bp, Artificial Sequence
  Oligonucleotide, Accession number NM 000765.1, Forward Primer (5`-3`)
ctatgatactgtgctacagt

SEQ ID NO: 28 - DNA, 20 bp, Artificial Sequence
  Oligonucleotide, Accession number NM 000765.1, Reverse Primer (5`-3`)
tcaggctccacttacggtct

SEQ ID NO: 29 - DNA, 20 bp, Artificial Sequence
  Oligonucleotide, Accession number AF208532, Forward Primer (5`-3`)
caagtgacctccctgctcat

SEQ ID NO: 30 - DNA, 20 bp, Artificial Sequence
  Oligonucleotide, Accession number AF208532, Reverse Primer (5`-3`)
ctgatctccccagaatcacc

SEQ ID NO: 31 - DNA, 21 bp, Artificial Sequence
  Oligonucleotide, Accession number NM 000779.1, Forward Primer (5`-3`)
tgaccatgtgcatcaaaggag

SEQ ID NO: 32 - DNA, 20 bp, Artificial Sequence
  Oligonucleotide, Accession number NM 000779.1, Reverse Primer (5`-3`)
aaagccattcttggagcgca

SEQ ID NO: 33 - DNA, 20 bp, Artificial Sequence
  Oligonucleotide, Accession number NM_000413, Forward Primer (5`- 3`)
cagctggacgtaagggactc

SEQ ID NO: 34 - DNA, 20 bp, Artificial Sequence
  Oligonucleotide, Accession number NM_000413, Reverse Primer (5`- 3`)
aacttgctggcgcaataaac

SEQ ID NO: 35 - DNA, 20 bp, Artificial Sequence
  Oligonucleotide, Accession number XM_012571, Forward Primer (5`- 3`)
gagctttccaagtggggaat

SEQ ID NO: 36 - DNA, 20 bp, Artificial Sequence
  Oligonucleotide, Accession number XM_012571, Reverse Primer (5`- 3`)
agcaaggcagatccacaagt

SEQ ID NO: 37 - DNA, 20 bp, Artificial Sequence
  Oligonucleotide, Accession number XM_005568, Forward Primer (5`- 3`)
cctccgtagtcaagatgaca

SEQ ID NO: 38 - DNA, 20 bp, Artificial Sequence
  Oligonucleotide, Accession number XM_005568, Reverse Primer (5`- 3`)
caaggcagccacaggtttca

SEQ ID NO: 39 - DNA, 20 bp, Artificial Sequence
  Oligonucleotide, Accession number XM_003904, Forward Primer (5`- 3`)
tatgccctggcttttgcaga

SEQ ID NO: 40 - DNA, 20 bp, Artificial Sequence
  Oligonucleotide, Accession number XM_003904, Reverse Primer (5`- 3`)
cagtcttcatcacttatcct

SEQ ID NO: 41 - DNA, 20 bp, Artificial Sequence
  Oligonucleotide, Accession number XM_001544, Forward Primer (5`- 3`)
agaggcctgtgtccaagcta

SEQ ID NO: 42 - DNA, 20 bp, Artificial Sequence
  Oligonucleotide, Accession number XM_001544, Reverse Primer (5`- 3`)
gttgttcagggcctcgttta

SEQ ID NO: 43 - DNA, 20 bp, Artificial Sequence
  Oligonucleotide, Accession number M32313, Forward Primer (5`-3`)
atgttcctcgtccactacgg

SEQ ID NO: 44 - DNA, 20 bp, Artificial Sequence
  Oligonucleotide, Accession number M32313, Reverse Primer (5`-3`)
gcctccccttggtattttgt

SEQ ID NO: 45 - DNA, 20 bp, Artificial Sequence
  Oligonucleotide, Accession number AH002979, Forward Primer (5`-3`)
tttgacactggttcgtccaa

SEQ ID NO: 46 - DNA, 20 bp, Artificial Sequence
  Oligonucleotide, Accession number AH002979, Reverse Primer (5`-3`)
ctgccagacaccagtcttga

SEQ ID NO: 47 - DNA, 20 bp, Artificial Sequence
  Oligonucleotide, Accession number J04111, Forward Primer (5`-3`)
gagcggaccttatggctaca

SEQ ID NO: 48 - DNA, 20 bp, Artificial Sequence
  Oligonucleotide, Accession number J04111, Reverse Primer (5`-3`)
ccgttgctggactggattat

SEQ ID NO: 49 - DNA, 20 bp, Artificial Sequence
  Oligonucleotide, Accession number XM_010429, Forward Primer (5`- 3`)
gtggaagctgcaaggtcttc

SEQ ID NO: 50 - DNA, 20 bp, Artificial Sequence
  Oligonucleotide, Accession number XM_010429, Reverse Primer (5`- 3`)
gttgttgtcgtgtccagcac

SEQ ID NO: 51 - DNA, 20 bp, Artificial Sequence
  Oligonucleotide, Accession number D00943, Forward Primer (5`-3`)
tactgagcagctaggagaag

SEQ ID NO: 52 - DNA, 20 bp, Artificial Sequence
  Oligonucleotide, Accession number D00943, Reverse Primer (5`-3`)
tctgccttgatctggttgaa

SEQ ID NO: 53 - DNA, 18 bp, Artificial Sequence
  Oligonucleotide, Accession number J02624, Forward Primer (5`-3`)
ggccaaggtcatccatga

SEQ ID NO: 54 - DNA, 18 bp, Artificial Sequence
  Oligonucleotide, Accession number J02624, Reverse Primer (5`-3`)
tcagtgtagcccaggatg

SEQ ID NO: 55 - DNA, 20 bp, Artificial Sequence
  Oligonucleotide, Accession number NM_021130, Forward Primer (5`- 3`)
cttgccattcctggacccaa

SEQ ID NO: 56 - DNA, 20 bp, Artificial Sequence
  Oligonucleotide, Accession number NM_021130, Reverse Primer (5`- 3`)
tttcgtgctctgagcactgg

SEQ ID NO: 57 - DNA, 21 bp, Artificial Sequence
  Oligonucleotide, Accession number X00469, Forward Primer (5`-3`)
ctggttctggatacccagctg

SEQ ID NO: 58 - DNA, 19 bp, Artificial Sequence
  Oligonucleotide, Accession number X00469, Reverse Primer (5`-3`)
cctagggttggttaccagg

SEQ ID NO: 59 - DNA, 21 bp, Artificial Sequence
  Oligonucleotide, Accession number X01031, Forward Primer (5`-3`)
gtcacctcagggaatgctgtg

SEQ ID NO: 60 - DNA, 21 bp, Artificial Sequence
  Oligonucleotide, Accession number X01031, Reverse Primer (5`-3`)
gttgacaatcttctcctgagg

SEQ ID NO: 61 - DNA, 20 bp, Artificial Sequence
  Oligonucleotide, Accession number NM_012940, Forward Primer (5`- 3`)
ctctcccccatccacatcta

SEQ ID NO: 62 - DNA, 20 bp, Artificial Sequence
  Oligonucleotide, Accession number NM_012940, Reverse Primer (5`- 3`)
agacgaacatgacccctacg

SEQ ID NO: 63 - DNA, 20 bp, Artificial Sequence
  Oligonucleotide, Accession number NM_012692, Forward Primer (5`- 3`)
cacagggcagctctatgaca

SEQ ID NO: 64 - DNA, 20 bp, Artificial Sequence
  Oligonucleotide, Accession number NM_012692, Reverse Primer (5`- 3`)
cagacccagcaaagaagagg

SEQ ID NO: 65 - DNA, 21 bp, Artificial Sequence
  Oligonucleotide, Accession number L00316, Forward Primer (5`-3`)
gagttcttctctgggttcctg

SEQ ID NO: 66 - DNA, 21 bp, Artificial Sequence
  Oligonucleotide, Accession number L00316, Reverse Primer (5`-3`)
actgtgggtcatggagagctg

SEQ ID NO: 67 - DNA, 21 bp, Artificial Sequence
  Oligonucleotide, Accession number U33173, Forward Primer (5`-3`)
ctgctgctgctgaaacacgtg

SEQ ID NO: 68 - DNA, 21 bp, Artificial Sequence
  Oligonucleotide, Accession number U33173, Reverse Primer (5`-3`)
ggatgacagcgatactatcac

SEQ ID NO: 69 - DNA, 20 bp, Artificial Sequence
  Oligonucleotide, Accession number M20131, Forward Primer (5`-3`)
ctcctcgtcatatccatctg

SEQ ID NO: 70 - DNA, 20 bp, Artificial Sequence
  Oligonucleotide, Accession number M20131, Reverse Primer (5`-3`)
gcagccaatcagaaatgtgg

SEQ ID NO: 71 - DNA, 20 bp, Artificial Sequence
  Oligonucleotide, Accession number U39943, Forward Primer (5`-3`)
ggcatgcccttaatcaaaga

SEQ ID NO: 72 - DNA, 20 bp, Artificial Sequence
  Oligonucleotide, Accession number U39943, Reverse Primer (5`-3`)
agcctcagcatctcctgaaa

SEQ ID NO: 73 - DNA, 18 bp, Artificial Sequence
  Oligonucleotide, Accession number X64401, Forward Primer (5`-3`)
atccgatatggagatcac

SEQ ID NO: 74 - DNA, 18 bp, Artificial Sequence
  Oligonucleotide, Accession number X64401, Reverse Primer (5`-3`)
gaagaagtccttgcttgc

SEQ ID NO: 75 - DNA, 19 bp, Artificial Sequence
  Oligonucleotide, Accession number X79320, Forward Primer (5`-3`)
cgacttggaacccatagac

SEQ ID NO: 76 - DNA, 21 bp, Artificial Sequence
  Oligonucleotide, Accession number X79320, Reverse Primer (5`-3`)
ggcttagggagatttgacatg

SEQ ID NO: 77 - DNA, 19 bp, Artificial Sequence
  Oligonucleotide, Accession number M57718, Forward Primer (5`-3`)
ggtgacaaagaactacagc

SEQ ID NO: 78 - DNA, 21 bp, Artificial Sequence
  Oligonucleotide, Accession number M57718, Reverse Primer (5`-3`)
agaggagtcttgacctgccag

SEQ ID NO: 79 - DNA, 20 bp, Artificial Sequence
  Oligonucleotide, Accession number NM_012851, Forward Primer (5`- 3`)
cctctttgcccactatcagc

SEQ ID NO: 80 - DNA, 20 bp, Artificial Sequence
  Oligonucleotide, Accession number NM_012851, Reverse Primer (5`- 3`)
ggagacaaatgagggctctg

SEQ ID NO: 81 - DNA, 17 bp, Artificial Sequence
  Oligonucleotide, Accession number NM_024391, Forward Primer (5`- 3`)
tgtgctggtgacaggtg

SEQ ID NO: 82 - DNA, 20 bp, Artificial Sequence
  Oligonucleotide, Accession number NM_024391, Reverse Primer (5`- 3`)
acggtcatgggaatgagttc

SEQ ID NO: 83 - DNA, 20 bp, Artificial Sequence
  Oligonucleotide, Accession number NM_054007, Forward Primer (5`- 3`)
cctccgtagtcaagatgaca

SEQ ID NO: 84 - DNA, 20 bp, Artificial Sequence
  Oligonucleotide, Accession number NM_054007, Reverse Primer (5`- 3`)
caaggcagccacaggtttca

SEQ ID NO: 85 - DNA, 20 bp, Artificial Sequence
  Oligonucleotide, Accession number NM_024392, Forward Primer (5`- 3`)
tatgccctggcttttgcaga

SEQ ID NO: 86 - DNA, 20 bp, Artificial Sequence
  Oligonucleotide, Accession number NM_024392, Reverse Primer (5`- 3`)
cagtcttcatcacttatcct

SEQ ID NO: 87 - DNA, 20 bp, Artificial Sequence
  Oligonucleotide, Accession number NM_012584, Forward Primer (5`- 3`)
attccagttggagccttcct

SEQ ID NO: 88 - DNA, 20 bp, Artificial Sequence
  Oligonucleotide, Accession number NM_012584, Reverse Primer (5`- 3`)
ccctcttcccatcattgaga

SEQ ID NO: 89 - DNA, 20 bp, Artificial Sequence
  Oligonucleotide, Accession number J05035, Forward Primer (5`-3`)
ccttcctgttctgcaccttc

SEQ ID NO: 90 - DNA, 20 bp, Artificial Sequence
  Oligonucleotide, Accession number J05035, Reverse Primer (5`-3`)
tgcaaacactacaccctgga

SEQ ID NO: 91 - DNA, 20 bp, Artificial Sequence
  Oligonucleotide, Accession number NM_012642, Forward Primer (5`- 3`)
ccgtggtcctcaccaactac

SEQ ID NO: 92 - DNA, 20 bp, Artificial Sequence
  Oligonucleotide, Accession number NM_012642, Reverse Primer (5`- 3`)
ttccacccacagttaccaca

SEQ ID NO: 93 - DNA, 20 bp, Artificial Sequence
  Oligonucleotide, Accession number X17163, Forward Primer (5`-3`)
cgaccagaacgatggacttt

SEQ ID NO: 94 - DNA, 20 bp, Artificial Sequence
  Oligonucleotide, Accession number X17163, Reverse Primer (5`-3`)
gggtaggacacccaaacaaa

SEQ ID NO: 95 - DNA, 20 bp, Artificial Sequence
  Oligonucleotide, Accession number M23264, Forward Primer (5`-3`)
ggtaatatccgaaggcagca

SEQ ID NO: 96 - DNA, 20 bp, Artificial Sequence
  Oligonucleotide, Accession number M23264, Reverse Primer (5`-3`)
cagcccagactctcaccttc

SEQ ID NO: 97 - DNA, 24 bp, Artificial Sequence
  Oligonucleotide, Accession number X15938, Forward Primer (5`-3`)
ggaagagcgagcggcgcatcaagg

SEQ ID NO: 98 - DNA, 22 bp, Artificial Sequence
  Oligonucleotide, Accession number X15938, Reverse Primer (5`-3`)
ctgctggacaggttattcctca

SEQ ID NO: 99 - DNA, 18 bp, Artificial Sequence
  Oligonucleotide, Accession number X02231, Forward Primer (5`-3`)
ggccaaggtcatccatga

SEQ ID NO: 100 - DNA, 18 bp, Artificial Sequence
  Oligonucleotide, Accession number X02231, Reverse Primer (5`-3`)
tcagtgtagcccaggatg

SEQ ID NO: 101 - DNA, 20 bp, Artificial Sequence
  Oligonucleotide, Accession number M19533, Forward Primer (5`-3`)
cttgccattcctggacccaa

SEQ ID NO: 102 - DNA, 20 bp, Artificial Sequence
  Oligonucleotide, Accession number M19533, Reverse Primer (5`-3`)
tttcgtgctctgagcactgg

SEQ ID NO: 103 - protein, 10 aa, Artificial Sequence
  synthetic decapeptide
AFASYKLKPA

(19) United States
(12) Patent Application Publication
(10) Pub. No.: US 2006/0258632 A1
(43) Pub. Date: Nov. 16, 2006

(54) Methods and agents for treating cardiovascular diseases

(75) Inventors: Jurgen Borlak, Lehrte/ OT Immensen (DE); Thomas Thum, Hannover (DE)

Correspondence Address: NORRIS, MCLAUGHLIN & MARCUS, P.A., 875 THIRD AVE, 18TH FLOOR, NEW YORK, NY, 10022, US

(73) Assignee: Fraunhofer-Gesellschaft zur Forderung der angewandten Forschung E.V., Munchen (DE)

(21) Appl. No.: 11/418,402

(22) Filed: May 4, 2006

Related U.S. Application Data
(62) Division of application No. 10/309,562, filed on Dec. 4, 2002, now abandoned.

(30) Foreign Application Priority Data
Sep. 4, 2002 (EP) 02090315.9

(51) Int. Cl. A61K 31/57

(52) U.S. Cl. 514/177

(57) ABSTRACT

This invention relates to a method and an agent for treating cardiovascular diseases, especially cardiac hypertrophy, wherein said method and agent consist in increasing testosterone concentration in pathological tissues to normal levels or inhibiting and/or eliminating metabolites from testosterone metabolism. The testosterone concentration in pathological tissues can be increased to normal levels by administering at least one substance from the following groups: testosterone; substances with effects similar to those of testosterone; testosterone mimetics; substances that enhance testosterone synthesis; substances that inhibit testosterone metabolism. Metabolites from testosterone metabolism can be inhibited and/or eliminated by administering at least one substance from the following groups: substances that bind to the androgen receptor, causing the receptor levels to be regulated and thus normalized; substances that bind to the androgen receptor, regulating the androgen receptor-mediated gene expression by inhibiting it, as is observed in cardiac hypertrophy. Areas of application are medicine and pharmaceuticals industry.

### FIELD OF THE INVENTION

This invention relates to methods and agents for treating cardiovascular diseases, especially cardiac hypertrophy. Areas of application are medicine and pharmaceuticals industry.

### BACKGROUND OF THE INVENTION

Testosterone is an androgen that is primarily produced in the Leydig cells of the testis. It can either act directly or, after modification, via enzymes, e.g., after reduction at position 5 of the steroid skeleton. Androgenic signaling (hormone effect) is responsible for the development of male sex characteristics and affects metabolic processes of the oil glands and accessory reproductive glands (e.g., prostate, seminal vesicles). These effects are primarily mediated by 5-alpha-dihydrotestosterone (5-alpha-DHT) (Rosenfield et al., 1998). Testosterone and numerous derivatives regulate spermatogenesis, recruit the substances required for spermatogenesis, including fructose, increase protein synthesis, promote bone growth and enhance libido (O'Donnell et al., 1999; Mason and Morris, 1997). Inside the prostate, 5-alpha-DHT may induce hypertrophy (Schroder, 1994); 5-alpha steroid reductase inhibitors are thus used for the treatment of prostate tumor patients to bring about reduction in size of the hypertrophic gland tissue (Schulman, 2001). At present, 5-alpha steroid reductase inhibitors are used to treat hormone-responsive alopecia, prostate hyperplasia and prostate cancer (Schulman, 2001).

Testosterone metabolism is regulated via different enzymatic pathways in the body, including the cytochrome P450 monooxygenase system, several 17-beta-hydroxy-steroid dehydrogenases, 3-beta-hydroxy-steroid dehydrogenase, aromatase and 5-alpha steroid reductase (FIG. 1). In most of the target organs, testosterone is thus reduced to 5-alpha DHT, a biologically highly active androgen, and, bound to cytoplasmatic receptors, transferred into the nuclei, where a hormone-receptor-chromatin complex is formed (Porto et al., 1995). This initiates an activation of specific genes, which in turn, induce mRNA synthesis and the ribosomal translation and, finally, leads to a de novo protein synthesis.

Both spermatogenesis and androgen secretion are controlled by hormones of the anterior lobe of the pituitary gland; together with androgens follicle-stimulating hormones (FSHs) promote spermatogenesis by supporting the function of Sertoli cells. Luteinizing hormones (LHs) affect the Leydig cells and stimulates androgen release. These secreted androgens have an inhibiting effect both on the release of FSHs and on that of LHs. The relatively slight inhibiting effect of these androgens is supported by estradiol, which is synthesized in the testis and converted from testosterone in the peripheral tissues. Injections of testosterone into the hypothalamus cause testicular atrophy and a decrease in secretion of a joint releasing hormone for FSH and LH.

Androgen release is strongly inhibited by estrogens and, to a lesser extent, by progestogens. Via a feedback mechanism, they inhibit the release of gonadotropic hormones. Lack of FSHs thus leads to an interruption of spermatogenesis, and deficiency of LHs stops androgens secretion. In other words, administration of estrogens to men has an effect similar to that of surgical castration, only that it is caused by hormones.

In order to avoid the effects of androgens, chemically synthesized antiandrogens are used as receptor blocker. Due to their structure, which is similar to that of testosterone, they can bind to the androgen receptor, inhibiting the signal transduction mediated by the androgen receptor. Known blockers are cyproteron and cyproteron acetate. Under the influence of antiandrogens, development of the seminal duct and its accessory glands as well as the penis is inhibited. In addition, differentiation of the normal phenotype into the male phenotype in the hypothalamus is blocked. In adult animals, antiandrogens inhibit spermatogenesis as well as seminal vesicle and cause prostate and oil gland atrophy. Furthermore, antiandrogens increase secretion of FSH- and LH-releasing hormones and thus also release of FSHs and LHs.

An endocrine disorder of the testes caused by adenomas of the Leydig cells is seldom and leads to Pubertas praecox in adolescents, i.e., an abnormally early puberty.

Indications for androgen therapy include general androgen deficiency, e.g., after castration, male climacteric, impotentia generandi due to oligozoospermia and impotentia coeundi.

Administration of anabolics (such as testosterone and dihydrotestosterone) causes increased muscle-building (Sinha-Hikim et al., 2002). Most of the molecular causes, however, remain unknown.

Multicentrical studies currently investigate the therapeutic benefit of testosterone substitution during “male menopause”. Interim analyses support an increase in bone and muscle mass and a decrease in body fat after testosterone replacement. In contrast, administration of 5-alpha-dihydrotestosterone caused a decrease of the testosterone plasma level as well as the LH and FSH levels.

The biosynthesis of testosterone and dihydrotestosterone is similar to that of adrenocortical steroids, i.e., via delta-5-pregnenolone from the cholesterol pathway. In humans, subsequent synthesis is predominantly carried out via hydroxylation (hydroxypregnenolone) and additional metabolism to become dihydroepiandrosterone (DHEA). In most of the target organs, testosterone is reduced to dihydrotestosterone and stereo- and regio-selectively oxidized or hydroxilated via complex oxidation products. Studies by Thum and Borlak (2000) on isolated heart muscle cells of the rat and subcellular fractions (microsomes) of the human heart have shown that testosterone is metabolized to different products (including oxidation, reduction, isomerization). Some of these metabolites, such as dihydrotestosterone, are capable of binding to androgen receptors. After interaction between ligand and receptor, the androgen receptor is translocated into the nucleus and, after relevant modifications, a non-covalent bond is established between the receptor and responsive elements of numerous target genes (Porto et al., 1995). Only little is known, however, about the interaction between different transcription factors [including hierarchy and network as well as protein-protein interactions] and the androgen receptor in transcriptional activation of androgen receptor-responsive genes. There are indications that a transient therapy of preterm and newly born infants with glucocorticoid dexametasone causes cardiac hypertrophy (Skelton et al., 1998). In addition, it is known that premenopausal women have a lower risk of cardiovascular disease. Estrogen might possibly have a cardio-protective effect, because this protection decreases after the menopause and is accompanied by a decrease in estrogen level. The to date worldwide largest study (Women Health Study) with more than 16000 postmenopausal women investigated the benefit of a hormone combined substitution therapy (estrogen plus progesterone). Surprisingly, this therapy led to an increase in the risk of cardiac diseases, apoplexy, and invasive breast cancer by 20% and of thrombosis by 50% (JAMA, 2002).

It has already been described earlier on that testosterone is metabolized in several organs and plays an important role in the differentiation of the male phenotype. Metabolites of testosterone include dihydrotestosterone, which can establish an effective bound to the androgen receptor, thus inducing its activation. Furthermore, it is known that the androgen receptor controls certain genes in the transcriptional regulation and thus directly influences the transcriptional activation of these genes. Also, as aforementioned, cytochrome P450-dependent mono-oxygenases have crucial functions in the steroid metabolism and are responsible for regio- and stereoselective hydroxylation. Here, tissue-specific metabolism via cytochrome P450 mono-oxygenases takes on a special role, especially as the different isoforms are not expressed ubiquitously so that metabolism, and thus also the metabolite profile, varies between different tissue types and organs. The effects of these metabolites therefore depend on the individual cell types and tissues, whereby the androgen receptor plays a special role. Consequently, the presence of the androgen receptor and the receptor levels are of crucial importance for the biological signal transfer mediated by androgen and androgen-receptor ligands.

Onset and development of cardiac hypertrophy is considered to be influenced by multiple factors, and different hypotheses are currently being discussed. On the one hand, overcompensation of heart muscle cells activates different receptors, such as G-protein-coupled and tyrosine kinase-dependent receptors, which then cause an intensified synthesis of proteins that damage the heart muscle. Furthermore, the production of growth factors increases, such as an increased secretion of fibroblastic growth factor and transforming growth factor beta, which in turn causes cellular hypertrophy of the heart muscle cells. The role of hormones in the development of cardiac hypertrophy, however, remains unknown.

The invention was based on the general aim of finding novel methods for treating cardiovascular diseases, especially cardiac hypertrophy. The objective of the invention is to provide agents with which cardiac hypertrophy can be effectively treated. Furthermore, these agents are to be indicated for treating other cardiovascular diseases.

The invention is based on numerous gene and protein expression studies. Comparative studies on explanted hearts of patients with differentially diagnosed cardiac hypertrophy and healthy hearts that had not been released for transplantation were conducted (eight patients with dilated cardiomyopathy (DCM); n=5 had a left ventricular assist device (LVAD) implanted for more than six months and n=3 patients had DCM combined with cardiac hypertrophy).

Quite unexpected and surprisingly, these studies confirm a markedly altered metabolism of testosterone in hypertrophic heart tissue. A highly significant increase in production of 5-alpha-dihydrotestosterone and additional P450-catalyzed stereo- and regio-selective oxidation/hydroxylation products were observed. In a comparison of normal with hypertrophic heart muscle tissue, the studies show that the expression of genes that code isoforms for different P450 mono-oxygenases, such as CYP2A6/7, CYP4A11, P450 aromatase, and also renin, is upregulated (FIG. 2). The highly significant induction of the P450 mono-oxygenase expression leads to an increased concentration of the translated P450 proteins, which can be made responsible for the altered testosterone metabolism. The investigations provide evidence for a dysfunction/excessive production of 5-alpha-dihydrotestosterone, androstenedione, and additional stereo- and regio-selective hydroxy-derivates in human hypertrophic heart tissue, which play a causal role in the biological signaling of disease development (either receptor-mediated or -independent, FIGS. 3 and 4). By inhibition of 5-alpha reductase with finasteride, production of dihydrotestosterone in the heart tissue is reduced and normalized (FIG. 3). The increased testosterone metabolism causes a marked decrease in the testosterone serum level and a statistically highly significant (>12-fold) induction of the androgen receptor-expression (see FIG. 5). The induction of the androgen receptor is a biological response to the continuously decreasing testosterone serum level in hypertrophic hearts.

Additional experiments on spontaneously hypertensive rats (SHR), an established model of pathological hypertrophy, support the findings obtained with human hypertrophic hearts (FIGS. 3, 5 and 6). The heart tissue of the SHR strain also showed a markedly altered testosterone metabolism, and a highly significant increase in stereo- and regio-selective testosterone metabolites was observed (FIG. 3). When compared to normotensive rats, a nearly 30-fold induction of the androgen receptor can be observed in the hypertrophic heart tissue of the pathological rats (FIG. 5). Similarly, the intensified testosterone metabolism also caused a decrease in testosterone serum levels in the heart muscle, and this lack of testosterone in the tissue induced an intensified expression of the androgen receptor. A lack of testosterone in tissue causes induction of the androgen receptor in the heart.

Further studies provide evidence for a strong reduction of the expression of alpha-MHC in pathological/hypertrophic heart tissue (FIG. 7). Alpha-MHC is responsible for coding a structure protein in the heart muscle and is thus already being used as a marker of diseases in differential diagnosis of cardiac hypertrophy in university hospitals. Bioinformatic analyses of the promotor region of the alpha-MHC-coded gene yield proof for the presence of several androgen receptor binding sites (FIG. 9). This indicates that testosterone itself, and also ligands activating the androgen receptor, control alpha-MHC gene expression. A decrease in testosterone serum level, characteristic of cardiac hypertrophy, however, leads to a weaker expression of alpha-MHC.

Based on these study results, the method and agents of the present invention for treating cardiovascular diseases, especially cardiac hypertrophy, are disclosed.

The invention is employed by the methods described in the claims.

The method for treating cardiovascular diseases, especially cardiac hypertrophy, consists in an increase in testosterone concentration in pathological tissues to normal levels or in inhibiting and/or eliminating metabolites from testosterone metabolism.

Testosterone concentration in pathological tissues can be increased to normal levels by administering at least one substance from the following groups:
testosterone;
substance with effects similar to those of testosterone
testosterone mimetics;
substances that enhance testosterone synthesis
substances that inhibit testosterone metabolism.

Metabolites of testosterone metabolism can be inhibited and/or eliminated by administering at least one substance from the following groups:
substances that bind to the androgen receptor, causing the receptor levels to be regulated and thus normalized;
substances that bind to the androgen receptor, regulating the androgen receptor-mediated gene expression by inhibiting it, as is observed in cardiac hypertrophy.

This invention comprises agents for treating cardiovascular diseases, especially cardiac hypertrophy, wherein said agents contain substances that increase testosterone concentration in pathological tissues to normal levels or inhibit and/or eliminate metabolites from testosterone metabolism.

The agents contain at least one substance from the following groups, which increase testosterone concentration in pathological tissue to normal levels:
testosterone, preferably as testosterone esters, 19-nortestosterone, testosterone propionate, testosterone undecanoate, mesterolone, fluoxymesterone;
substances with effects similar to those of testosterone, preferably nandrolone decanoat, clostebole acetate, methenolone acetate, androstendione, dehydroepiandrosterone;
testosterone mimetics;
substances that enhance testosterone synthesis, preferably synthetic analogue gonadoliberine and additional non-steroid substances that promote an increased testosterone release as well as antagonists of hormones inhibiting gonadotropine-releasing hormones and lutenizing hormone-releasing hormones (LHRH);
substances that inhibit testosterone metabolism, especially synthesis of dihydrotestosterone; substances preferred include:
   a) cytochrome P450 (CYP) mono-oxygenase inhibitors; and/or
   b) steroid reductase inhibitors; and/or
   c) isomerase inhibitors; and/or
   d) 17-beta-hydroxy-steroid-dehydrogenase inhibitors; and/or
   e) 3-beta-hydroxy-steroid-dehydrogenase inhibitors; and/or
   f) aromatase inhibitors.

As synthetic analogue gonadoliberines are preferably used: Buserelin, Goserelin, Leuprorelin, Navarelin, Triptorelin; as antagonists of hormones inhibiting gonadotropine-releasing hormones are preferably used Abarelix, Antarelix, Cetrorelix, Ganirelix Acetat, Iturelix; and as antagonists of hormones inhibiting lutenizing hormone-releasing hormones (LHRH) are preferably used teverelix, a synthetic decapeptide that contains five aminoacids in D-configuration with the sequence Ac-D-Nal-D-Cpa-D-Pal-Ser--Tyr-D-Hci-Leu--Lys-(iPr)-Pro-D-Ala-NH2 (C47H100ClN15O14).

Substances to be used as cytochrome P450 (CYP) mono-oxygenase inhibitors include: amiodarone, cimetidine, fluoroquinolone, fluvoxamine, furafylline, methoxsalen, mibefradile, ticlopidine, thiotepa, cimetidine, felbamate, fluoxetine, fluvoxamine, indomethacine, ketoconazole, lansoprazole, modafinil, omeprazole, paroxetine, topiramate, fluconazole, fluvastatin, isoniazide, lovastatine, paroxetine, phenylbutazone, probenecid, sertraline, sulfamethoxazole, sulfaphenazole, teniposide, trimethoprime, zafirlukast, clecoxib, chlorpromazine, chlorpheniramine, clomipramine, cocaine, doxorubicine, halofantrine, red-haloperidol, levomepromazine, metoclopramide, methadone, mibefradil, moclobemide, quinidine, ranitidine, ritonavir, terbinafine, diethyl dithiocarbamate, disulfiram, delavirdine, indinavir, nelfinavir, ritonavir, saquinavir, ciprofloxacin, clarithromycin, diltiazem, erythromycin, gestodene, grapefruit juice, itraconazole, mifepristone, nefazodone, norfloxacin, norfluoxetine, or troleandomycin.

In addition, the invention can be realized with substances that inhibit the expression of genes coding for cytochrome P450 (CYP) mono-oxygenase isoforms, such as CYP2A6/7, CYP4A11, CYP2J2, or P450 aromatase.

5-alpha steroid reductase inhibitors, especially finasteride, are especially suitable as steroid reductase inhibitors.

Cyproterone acetate is preferably used as isomerase inhibitor.

Agents preferred as 17-beta-hydroxy-steroid-dehydrogenase inhibitors include 3beta-peptido-3alpha-hydroxy-5alpha-androstane-17-on-derivates, substances with a 17-spiro-gamma-lactone group, and 3beta-[(N-adamantylmethyl-N-butanoyl)aminomethyl]-3 alpha-hydroxy-5-alpha-androstane-17-on.

Cyproterone acetate is preferably used as 3-beta-hydroxy-steroid-dehydrogenase inhibitor, and tamoxifen, fadrozole, aminoglutethimide, formestane, testolactone, and 1,4,5-androstene-3,17-dione are preferably used as aromatase inhibitor.

In addition, the agents of this invention may contain one substance of the following groups, which inhibit and/or eliminate the metabolites of the testosterone metabolism and their effects:
substances that bind to the androgen receptor, causing the receptor levels to be regulated and thus normalized;
substances that bind to the androgen receptor, regulating the androgen receptor-mediated gene expression by inhibiting it, as is observed in cardiac hypertrophy.

Here, the use of antagonists and/or ligands of the androgen receptor is preferred.

Preferred antagonist of the androgen receptor are flutamide and bicalutamide, and preferred ligands of the androgen receptor include chiral and non-chiral analogue bicalutamides, chiral and non-chiral analogue bicalutamides with a trifluoride methyl group, and chiral and non-chiral analogue bicalutamides with a sulfide group.

Furthermore, the agents of the invention may contain alpha-MHC and/or substances that increase the expression of alpha-MHC.

In the following, the invention is further illustrated by reference to examples and figures.

Study Description

General

Animal Experiments

Male normotensive Sprague-Dawley and spontaneously hypertensive rats (SHR, weighting approx. 200 g) by Charles River (Sulzfeld, Germany) were used. Before surgery, the animals were anesthetized with intreaperitoneal administration of ketamin (anesthetic; 0.1 ml/100 g body weight), xylazin-hydrochloride (muscle relaxant; 0.05 ml/10 g body weight) and heparin (2000 i.e. heparin).

Isolation of Total RNA and Reverse Transcription into cDNA

Total RNA was isolated from heart tissue using the Total RNA Isolation Kit (Macherey-Nagel, Germany) according to the manufacturer's instructions. The quality of the isolated RNA was assessed by evaluating the ribosomal bands on agarose gel (1%). Of each sample, 2 μg RNA was used for reverse transcription. RNA and random primer (Roche Diagnostics, Mannheim, Germany) were preheated at 70° C. for 10 min. and subsequently incubated with 5×RT-AMV buffer (Roche Diagnostics, Mannheim, Germany), dNTPs (1 mM; Roche Diagnostics, Mannheim, Germany), 40U RNAse inhibitor (Stratagene, Amsterdam, Netherlands) and 20U AMV-RT (Promega, Mannheim, Germany) at a total volume of 20 μl for 60 min. at 42° C. Next, the added enzymes were inactivated by denaturation for 5 minutes at 95° C.

Thermocycler RT Polymerase Chain Reaction (PCR)

PCR reactions were carried out in a thermocycler (T3, Biometra, Germany) under the following conditions: Denaturation at 94° C. for 30 s, annealing at 57° C. for 60 s and extension at 72° C. for 60 s to amplify the cytochrome P450 isoforms, alpha-MHC (33 cycles each) and cyclophilin (26 cycles). Annealing temperature for detecting the androgen receptor, 17beta-hydroxy-steroid-dehydrogenase 1-4,3-beta-hydroxy-steroid-dehydrogenase 1, P450 aromatase, renin, c-jun and 5-alpha reductase was 55° C. for 60 s, and 30-35 PCR cycles were carried out.

Possible contamination with genomic DNA was prevented by direct addition of RNA extracts to the PCR cocktail. The PCR reactions were made in the linear amplification range. Amplificates were separated onto a 1.5% agarose gel, stained with ethidium bromide and photographed on a transilluminator (Kodak Image Station 440). Quantification was achieved using the program Kodak 1D 3.5 Network. Tables 1A and B depict the sequences of the oligonucleotides used for amplifying the different genes.

Quantitative Real-Time RT PCR

The studies were conducted With a lightcyler (Roche Diagnostics, Mannheim, Germany). One μl of the cDNA diluted at a ratio of 1:10 was mixed with 0.5 μM of the specific primer, 4 mM MgCl₂, and 2 μl Master-SYBR-Green PCR Mix (Roche Diagnostics, Mannheim, Germany) and adapted to a final volume of 20 μl. After initial denaturation at 95° C. for 30 s, PCR reaction was started at 55° C.-58° C. (annealing) for 7 s, followed by an extension phase of 12-20 s at 72° C. and a subsequent denaturation phase of 1 s at 95° C. Table 1 shows the oligonucleotide sequences. By the end of each extension phase, fluorescence was measured to be used for evaluation. To guarantee that no unspecific primer dimers are measured, fluorescence detection was conducted above the temperature characteristic of primer dimers (as assessed in preliminary studies). Amplification was followed by a melting curve analysis, measuring fluorescence by slowly increasing the temperature of the DNA synthesis products from 65° C. to 95° C. (0.1° C./s). The exact amount of the obtained PCR product was determined by co-amplification of standardized cardiac cDNA and according to the manufacturer's instructions.

### EXAMPLE 1

In cell culture experiments with isolated heart muscle cells from the rat, addition of testosterone to the culture medium reduces and normalizes androgen receptor expression (FIG. 5c).

### EXAMPLE 2

Experiments on rat heart muscle cells provide evidence for the therapeutic effect of a testosterone substitution, causing a normalization of alpha-MHC expression (FIGS. 7 and 8).

### EXAMPLE 3

An established cardiac hypertrophy starts to regress after, e.g., implantation of a left ventricular assist device (LVAD). Studies with LVAD implantation tissue are especially valuable because the patients are first differentially diagnosed with cardiac hypertrophy which, due to the assist device, regresses after considerable time has passed, normalizing the heart's function. Therefore, testosterone metabolism in LVAD-supported hearts and in patients under conventional treatment was investigated. When comparing healthy patients with those with LVAD implants, metabolism of testosterone into different metabolites (5-alpha-DHT, different hydroxylation products) in hypertrophic heart was markedly increased (FIG. 3). We therefore provide evidence for a causal relationship between cardiac hypertrophy and an altered steroid metabolism of testosterone.

### EXAMPLE 4

It is shown that decreased testosterone metabolism in healthy and LVAD-treated heart tissue is coupled with a decreased expression of different cytochrome P450 mono-oxygenases (CYP2A6/7, CYP2J2, CYP4A11) (FIG. 2).

In addition, when compared to hypertrophic hearts, the genes c-jun, renin, 5-alpha reductase and the expression of the androgen receptor are significantly repressed in LVAD-supported hearts (FIGS. 2 and 10), while expression of alpha-MHC in healthy and LVAD-supported heart tissue, which leads to reduced intraventricular pressure, is greater (FIG. 7). This proofs that the dysfunctioning steroid metabolism in cardiac hypertrophy can be normalized after LVAD implantation and that a regress of cardiac hypertrophy after LVAD implantation can also be clinically diagnosed using echocardiography (Zafeiridis et al., 1998).

In summary, steroid metabolism in human hypertrophic hearts and, in addition, also in pathological rat models is significantly altered, a finding that came unexpectedly and surprisingly. The experiments that have led to the invention document a pathological induction of CYP mono-oxygenases and enzymes involved in testosterone metabolism and studies with tissues and subcellular fractions from biopsy materials of hypertrophic and LVAD-implanted heart tissues show that the investigations are of clinical relevance. Studies on isolated heart muscle cells provide clear evidence for a therapeutic effect of testosterone itself, as has been shown with the example of the alpha-MHC-coded gene. The repression of the alpha-MHC gene expression observed during the clinical study correlated well with cardiac hypertrophy and has thus been accepted as biological marker. Further studies on explanted heart and experimental investigations in the rat model provide evidence for a deregulation of the androgen receptor in the hypertrophic heart. The induction of the androgen receptor observed in the hypertrophic heart is a biological response/adaptation to the markedly decreased testosterone level in the heart tissue. Testosterone and 5-alpha-dihydrotestosterone activate the androgen receptor differently, regulating different androgen receptor-responsive genes. Thus, due to the different ligands of the receptor (testosterone versus dihydrotestosterone), different target genes are activated. In the case of cardiac hypertrophy, less receptor is present to bind to the responsive elements of the alpha-MHC gene's promoter, thus deregulating alpha-MHC. A decreased expression of alpha-MHC, however, leads to negative inotropism and lower cardiac output, as is clinically observed in advanced cardiac hypertrophy and cardiac insufficiency.

TABLE 1A
Used oligonucleotides in experiments with human tissues

| | | | | |
|---|---|---|---|---|
| Accession | | | | Product |
| number | Gene | Forward Primer (5′-3′) | Reverse Primer (5′-3′) | length (bp) |

| | | | | |
|---|---|---|---|---|
| | Cytochrome | | | |
| | P450 genes | | | |
| D01150 | CYP 1A1 | TCACAGACACAGCCTGATTGAG | GATGGGTTGACCCATAGCTT | 432 |
| M38504 | CYP 1A2 | TGGCTTCTACATCCCCAAGAAAT | TTCATGGTCAGCCCGTAGAT | 308 |
| U22027 | CYP 2A6/7 | GTGTGGACATGATGCCGT | AGGACTTGAGGCGGAAGT | 1151 |
| X13494 | CYP 2B6/7 | CCATACACAGAGGCAGTCAT | GGTGTCAGATCGATGTCTTC | 357 |
| XM_050922 | CYP 2C8 | GATCATGTAATTGGCAGACACA | CCTGCTGAGAAAGGCATGAAG | 311 |
| XM_050918 | CYP 2C9 | AGCTTGGAAAACACTGCAGT | CCTGCTGAGAAAGGCATGAAG | 437 |
| NM_000772 | CYP 2C18 | CTGTAACTGATATGTTTGGG | CCTGCTGAGAAAGGCATGAAG | 431 |
| NM_000769 | CYP 2C19 | GTAATCACTGCAGCTGACTTAC | CCTGCTGAGAAAGGCATGAAG | 431 |
| M33189 | CYP 2D6 | TGATGAGAACCTGCGCATAG | ACCGATGACAGGTTGGTGAT | 332 |
| AF084225 | CYP 2E1 | AGCACAACTCTGAGATATGG | ATAGTCACTGTACTTGAACT | 365 |
| J02906 | CYP 2F1 | ATGAACTTGCCGCACCGCGT | ACAGGCTCCACTTACGGTGC | 283 |
| X12387 | CYP 3A4 | CCAAGCTATGCTCTTCACCG | TCAGGCTCCACTTACGGTGC | 323 |
| L26985 | CYP 3A5 | TGTCCAGCAGAAACTGCAAA | TTGAAGAAGTCCTTGCGTGTC | 472 |
| NM_000765.1 | CYP 3A7 | GTATGATAGTGTGCTAGAGT | TCAGGCTCCACTTACGGTCT | 474 |
| AF208532 | CYP 4A11 | GAAGTGAGGTGGGTGCTGAT | GTGATGTGGGGAGAATGAGG | 280 |
| NM_000779.1 | CYP 4B1 | TGAGGATGTGGATGAAAGGAG | AAAGCGATTGTTGGAGGGGA | 397 |
| | 17 beta | | | |
| | hydroxy-steroid- | | | |
| | dehydrogenase | | | |
| | genes | | | |
| NM_000413 | 17b-HSD I | CAGCTGGACGTAAGGGACTC | AACTTGCTGGCGCAATAAAC | 293 |
| XM_012571 | 17b-HSD II | GAGCTTTCCAAGTGGGGAAT | AGCAAGGCAGATCCACAAGT | 299 |
| XM_005568 | 17b-HSD III | GGTGGGTAGTGAAGATGAGA | CAAGGCAGCCACAGGTTTCA | 333 |
| XM_003904 | 17b-HSD IV | TATGCCCTGGCTTTTGCAGA | GAGTGTTGATGAGTTATGGT | 275 |
| XM_001544 | 3 beta hydroxy- | AGAGGCCTGTGTCCAAGCTA | GTTGTTCAGGGCCTCGTTTA | 298 |
| | steroid-dehydro- | | | |
| | genase I | | | |
| M32313 | 5 alpha reductase | ATGTTCTCGTCCACTACGG | GCCTCCCCTTGGTATTTTGT | 300 |
| | Marker genes for | | | |
| | cardiac hypertrophy | | | |
| AH002979 | renin | TTTGACACTGGTTCGTCCAA | CTGCCAGACACCAGTCTTGA | 498 |
| J04111 | c-jun | GAGCGGACCTTATGGCTACA | CCGTTGCTGGACTGGATTAT | 192 |
| XM_010429 | Androgen receptor | GTGGAAGCTGCAAGGTCTTC | GTTGTTGTCGTGTCCAGCAC | 350 |
| D00943 | alpha MHC | TACTGAGCAGCTAGGAGAAG | TCTGCCTTGATCTGGTTGAA | 177 |
| | Housekeeping | | | |
| | genes | | | |
| J02624 | GAPDH | GGCCAAGGTCATCCATGA | TCAGTGTAGCCCAGGATG | 353 |
| NM_021130 | cyclophilin | CTTGCCATTCCTGGACCCAA | TTTCGTGCTCTGAGCACTGG | 347 |

TABLE 1B
Used oligonucleotides in experiments with rat tissues

| | | | | |
|---|---|---|---|---|
| Accession | | | | Product length |
| number | Gene | Forward Primer (5′-3′) | Reverse Primer (5′-3′) | (bp) |

| | | | | |
|---|---|---|---|---|
| | Cytochrome | | | |
| | P450 genes | | | |
| X00469 | CYP 1A1 | CTGGTTCTGGATACCCAGCTG | CCTAGGGTTGGTTACCAGG | 331 |
| X01031 | CYP 1A2 | GTCACCTCAGGGAATGCTGTG | GTTGACAATCTTCTCCTGAGG | 236 |
| NM_012940 | CYP 1B1 | CTCTCCCCCATCCACATCTA | AGACGAACATGACCCCTACG | 350 |
| NM_012692 | CYP 2A1/2 | CACAGGGCAGCTCTATGACA | CAGACCCAGCAAAGAAGAGG | 275 |
| L00316 | CYP 2B1/2 | GAGTTCTTCTCTGGGTTCCTG | ACTGTGGGTCATGGAGAGCTG | 549 |
| U33173 | CYP 2C11 | CTGCTGCTGCTGAAACACGTG | GGATGACAGCGATACTATCAC | 248 |
| M20131 | CYP 2E1 | CTCCTCGTCATATCCATCTG | GCAGCCAATCAGHAAATGTGG | 473 |
| U39943 | CYP 2J3 | GGCATGCCCTTAATCAAAGA | AGCCTCAGCATGTCCTGAAA | 364 |
| X64401 | CYP 3A1 | ATCCGATATGGAGATCAC | GAAGAAGTCCTTGCTTGC | 579 |
| X79320 | CYP3A2 | CGACTTGGAACCCATAGAC | GGCTTAGGGAGATTTGACATG | 116 |
| M57718 | CYP 4A1 | GGTGACAAAGAACTACAGC | AGAGGAGTCTTGACCTGCCAG | 344 |
| | 17 beta hydroxy- | | | |
| | steroid-dehydro- | | | |
| | genase genes | | | |
| NM_012851 | 17b-HSD I | CCTCTTTGCCCACTATCAGC | GGAGACAAATGAGGGCTCTG | 299 |
| NM_024391 | 17b-HSD II | TGTGCTGGTGACAGGTG | ACGGTCATGGGAATGAGTTC | 295 |
| NM_054007 | 17b-HSD III | CCTCCGTAGTCAAGATGACA | CAAGGCAGCCACAGGTTTCA | 333 |
| NM_024392 | 17b-HSD IV | TATGCCCTGGCTTTTGCAGA | CAGTCTTCATCACTTATCCT | 275 |
| NM_012584 | 3 beta hydroxy- | ATTCCAGTTGGAGCCTTCCT | CCCTCTTCCCATCATTGAGA | 292 |
| | steroid-dehydro- | | | |
| | genase I | | | |
| J05035 | 5 alpha reductase | CCTTCCTGTTCTGCACCTTC | TGCAACACTACACCCTGGA | 308 |
| | Marker genes for | | | |
| | cardiac hypertrophy | | | |
| NM_012642 | renin | CCGTGGTCCTCACCAACTAC | TTCCACCCACAGTTACCACA | 279 |
| X17163 | c-jun | CGACCAGAACGATGGACTTT | GGGTAGGACACCCAAACAAA | 347 |
| M23264 | Androgen receptor | GGTAATATCCGAAGGCAGCA | CAGCCCAGACTCTCACCTTC | 381 |
| X15938 | alpha MHC | GGAAGAGCGAGCGGCGCATCAAGG | CTGCTGGACAGGTTATTCCTCA | 334 |
| | Housekeeping genes | | | |
| X02231 | GAPDH | GGCCAAGGTCATCCATGA | TCAGTGTAGCCCAGGATG | 353 |
| M19533 | cyclophilin | CTTGCCATTCCTGGACCCAA | TTTCGTGTCTGAGCACTGG | 347 |

### REFERENCES

[No authors listed] Risks and benefits of estrogen plus progestin in healthy postmenopausal women: principal results From the Women's Health Initiative randomized controlled trial. JAMA. 2002 Jul. 17; 288(3):321-33.
Arlotto M P, Trant J M, Estabrook R W. Measurement of steroid hydroxylation reactions by high-performance liquid chromatography as indicator of P450 identity and function. Methods Enzymol. 1991; 206:454-62.
Claessens F, Verrijdt G, Schoenmakers E, Haelens A, Peeters B, Verhoeven G, Rombauts W. Selective DNA binding by the androgen receptor as a mechanism for hormone-specific gene regulation. J Steroid Biochem Mol. Biol. 2001 January-March; 76(1-5):23-30.
Cleutjens K B, van Eekelen C C, van der Korput H A, Brinkmann A O, Trapman J. Two androgen response regions cooperate in steroid hormone regulated activity of the prostate-specific antigen promoter. J Biol. Chem. 1996 Mar. 15; 271 (11):6379-88.
Karvonen U, Kallio P J, Janne O A, Palvimo J J. Interaction of androgen receptors with androgen response element in intact cells. Roles of amino- and carboxyl-terminal regions and the ligand. J Biol. Chem. 1997. Jun. 20; 272(25):15973-9.
Mason R A, Morris H A. Effects of dihydrotestosterone on bone biochemical markers in sham and oophorectomized rats. J Bone Miner Res. 1997 Sep.; 12(9):1431-7.
O'Donnell L, Pratis K, Stanton P G, Robertson D M, McLachlan R I. Testosterone-dependent restoration of spermatogenesis in adult rats is impaired by a 5alpha-reductase inhibitor. J Androl. 1999 Jan.-Feb.; 20(1):109-17.
Porto C S, Lazari M F, Abreu L C, Bardin C W, Gunsalus G L. Receptors for androgen-binding proteins: internalization and intracellular signalling. J Steroid Biochem Mol. Biol. 1995 June; 53(1-6):561-5.
Rosenfield R L, Deplewski D, Kentsis A, Ciletti N. Mechanisms of androgen induction of sebocyte differentiation. Dermatology. 1998; 196(1):43-6.
Schroder F H. 5 alpha-reductase inhibitors and prostatic disease. Clin Endocrinol (Oxf). 1994 August; 41(2):139-47.
Schulman C C. Long-term aspects of medical treatment of BPH. Eur Urol. 2001; 40 Suppl 3:8-12.
Sinha-Hikim I, Artaza J, Woodhouse L, Gonzalez-Cadavid N, Singh A B, Lee M I, Storer T W, Casaburi R, Shen R, Bhasin S. Testosterone-induced increase in muscle size in healthy young men is associated with muscle fiber hypertrophy. Am J Physiol Endocrinol Metab. 2002 July; 283(1):E154-64.
Skelton, R., Gill, A. B. and Parsons, J. M. (1998) Cardiac effects of short course dexamethasone in preterm infants. Arch Dis Child Fetal Neonatal Ed 78, 133-7.
Smith P K, Krohn R1, Hermanson G T, Mallia A K, Gartner F H, Provenzano M D, Fujimoto E K, Goeke N M, Olson B J, Klenk D C. Measurement of protein using bicinchoninic acid. Anal Biochem. 1985 October; 150(1):76-85.
Thum T, Borlak J. Cytochrome P450 mono-oxygenase gene expression and protein activity in cultures of adult cardiomyocytes of the rat. Br J. Pharmacol. 2000a Aug; 130(8): 1745-52.
Thum T, Borlak J. Gene expression in distinct regions of the heart. Lancet. 2000b March 18; 355(9208):979-83.
Zafeiridis A, Jeevanandam V, Houser S R, Margulies K B. Regression of cellular hypertrophy after left ventricular assist device support. Circulation. 1998 Aug. 18; 98(7):656-62.

### FIGURES

FIG. 1) Scheme of deregulated testosterone metabolism in human hypertrophic heart.

FIG. 2) Semiquantitative real-time RT-PCR on cardiac expression of different genes in healthy, hypertrophic and LVAD-supported (left ventricular assist device) human heart. For details refer to “Study Description, General” and Table 1 A.

FIG. 3) Testosterone metabolism in human and rat heart microsomal suspensions. After adding 5-alpha-steroid reductase inhibitor finasteride (25 μM), 5-alpha-DHT production decreased by 60%.

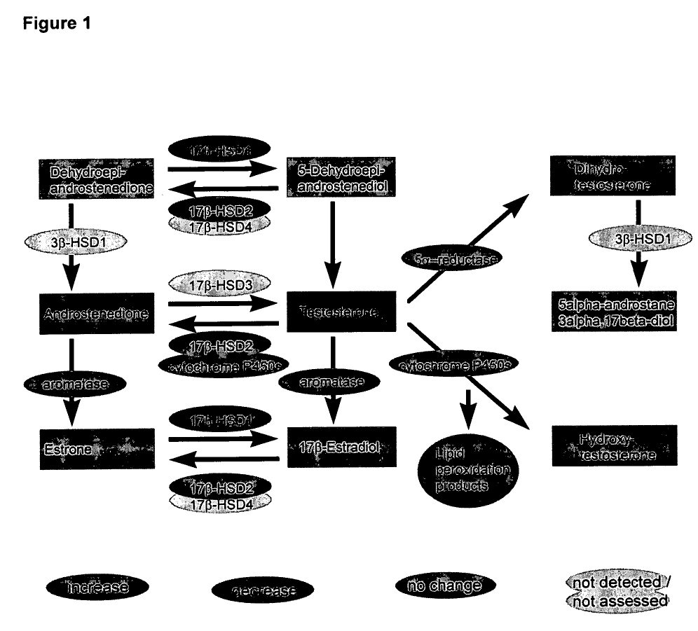

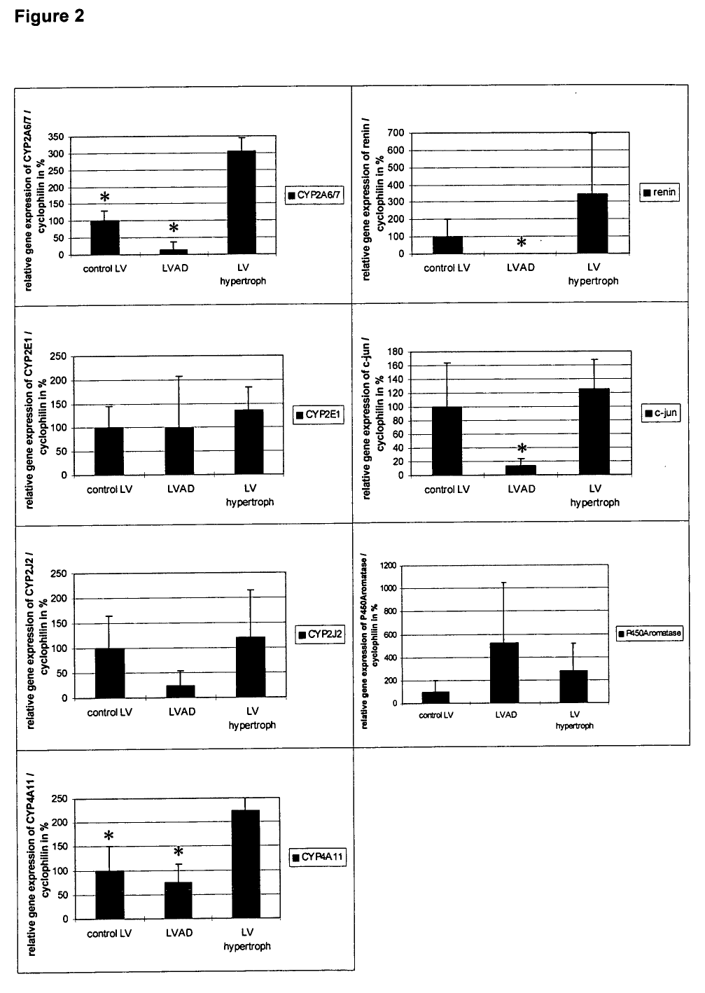

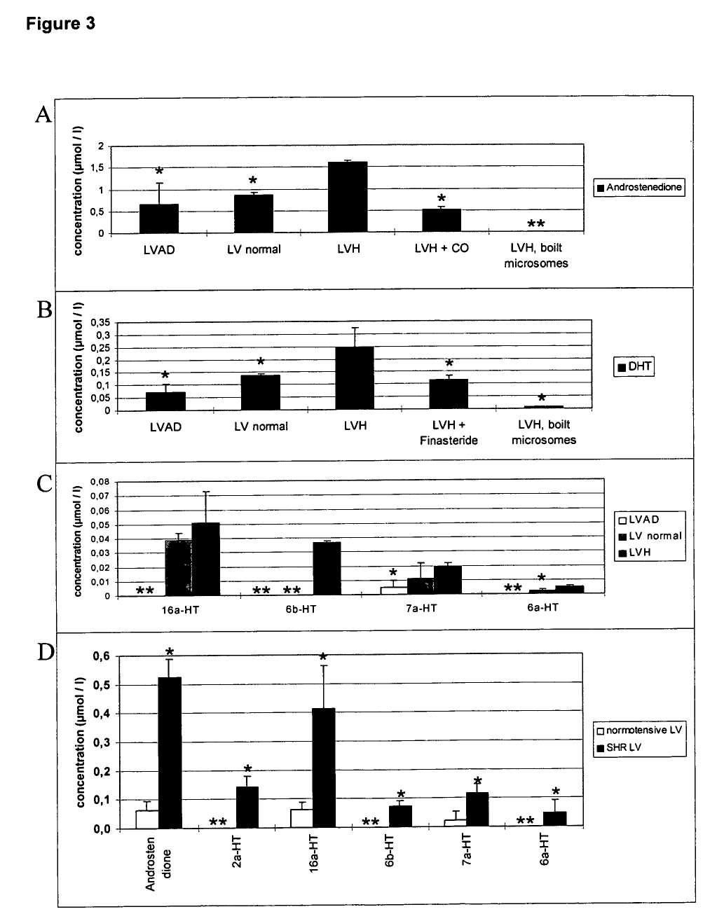

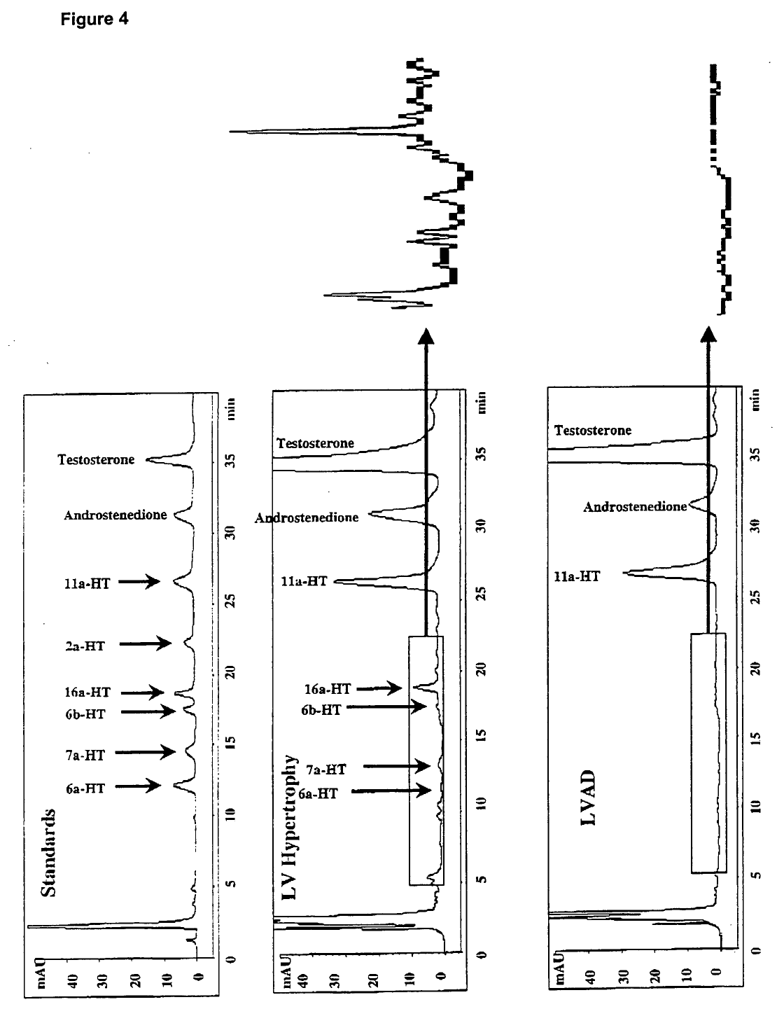

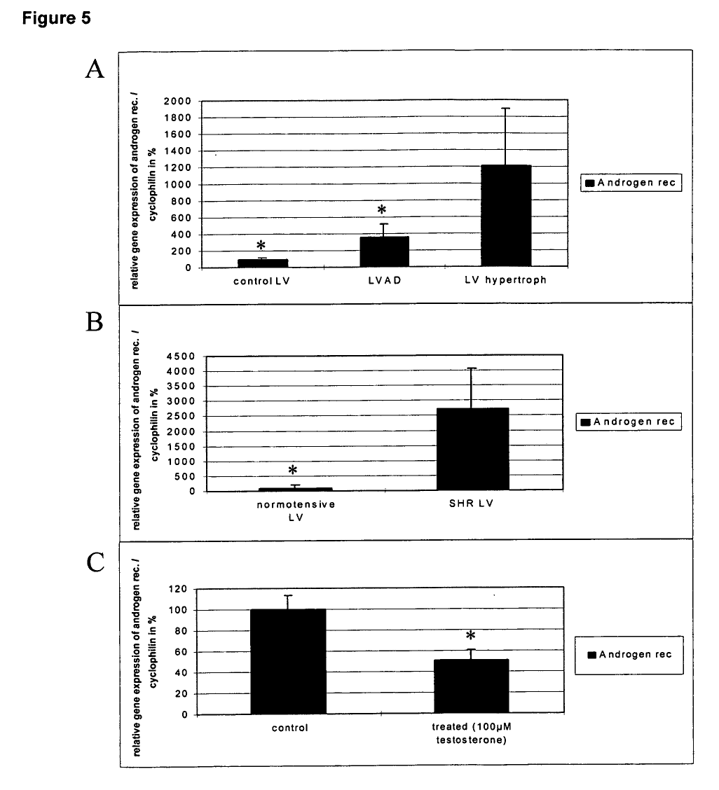

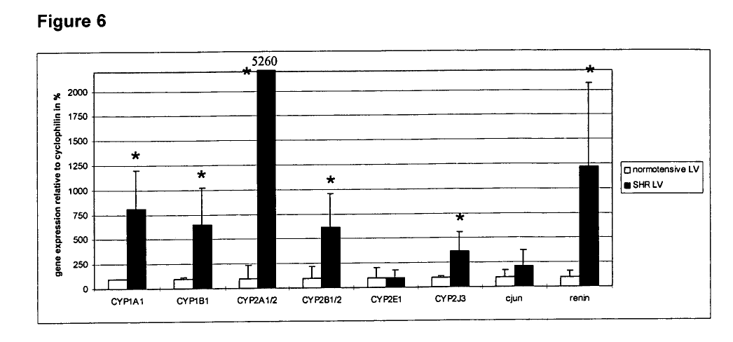

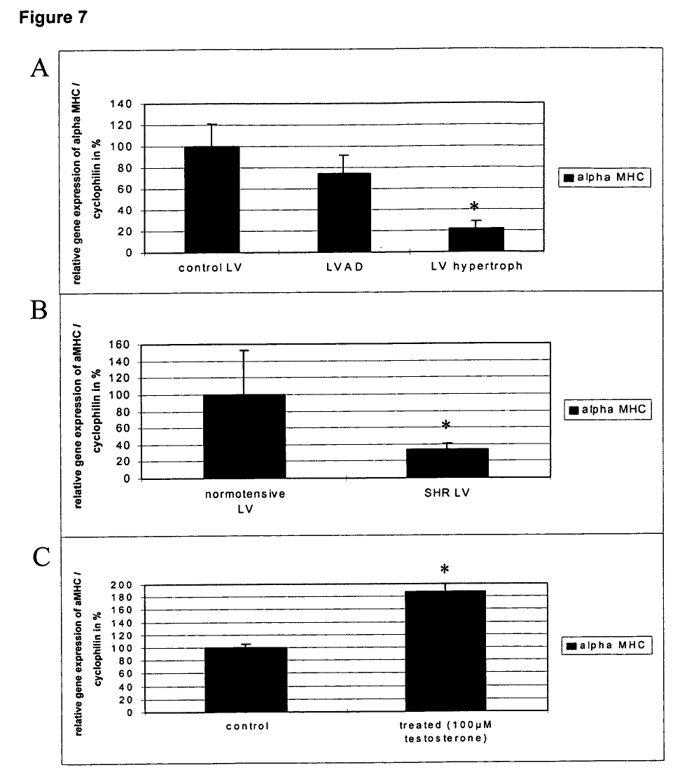

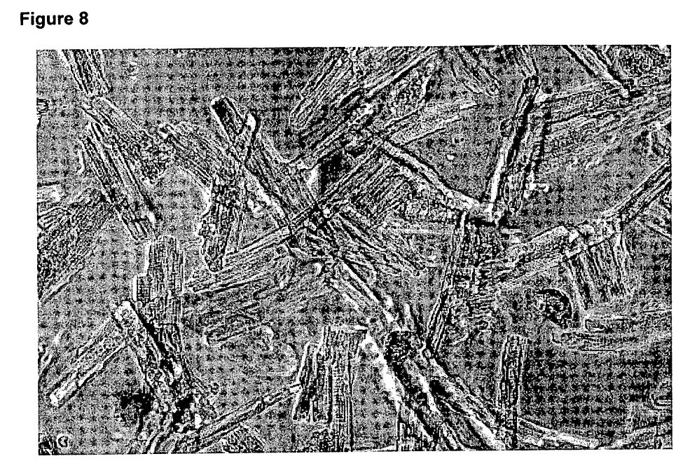

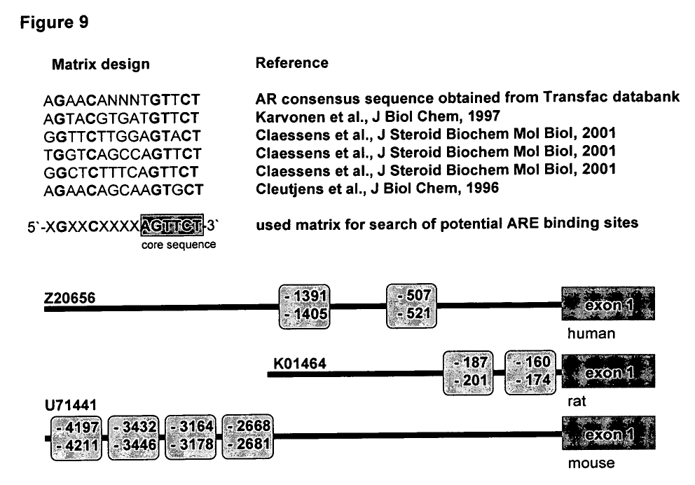

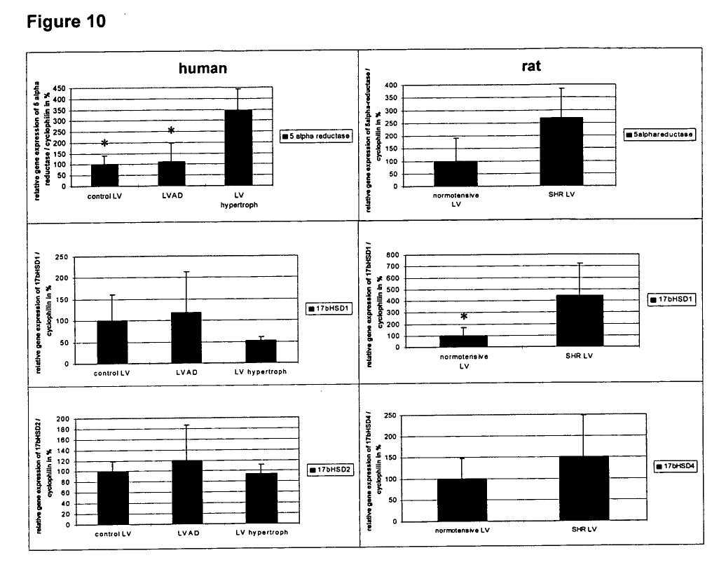

Microsomal membranes were isolated from ventricular tissue as follows:

Tissues from human and rat heart (ventricles) were cut into small (approx. 1 mm³) pieces and homogenized in an ultraturrax (IKA, Germany) in KCl-buffer (0.15M, pH 7.4) at 4° C. Subsequent isolation was carried out as described by Thum and Borlak (2000b). The microsomal suspension was transferred to a TRIS-sucrose buffer (0.25M sucrose, 20 mM TRIS-buffer, 5 mM EDTA) and stored at −80° C. until further use.

Protein concentrations in the isolated heart microsomes were determined according to Smith et al. (1985) and adjusted to 1 mg/ml.

In a shaking water bad, 500 μg microsomal protein was incubated with 100 μM testosterone and 1 mg/ml NADPH at a total volume of 1 ml TRIS buffer (20 mM, Sigma, Deisenhofen, Germany) for 4 h at 37° C. Samples were then shock-frozen in liquid nitrogen and stored at −80° C. until further analysis. Prior to incubation, 25 μM finasteride was added to some of the samples.

FIG. 4) HPLC (high pressure liquid chromatography) chromatograms of testosterone metabolism (100 μM) in microsomal suspensions of hypertrophic hearts and hearts with left ventricular assist devices (LVAD).

Testosterone and its metabolites were analyzed with HPLC according to a slightly altered method by Arlotto et al. (1991). Prior to sample analysis, 11-alpha-hydroxy-progestin (1 μg) was added as internal standard. After adding 100 μl isopropanol to 1 ml of the sample to be analyzed, the sample was extracted by shaking (20 min) with 5 ml ethyl acetate. The extracts were evaporated to dryness using nitrogen and reconstituted in 100 μl of the mobile HPLC phase (water/methanol/acetonitrile, 60/25/15, v/v/v). Next, 80 μl of the sample were injected into the HPLC system (Hewlett Packard HP1100). The flow rate was 1 ml min. Chromatographic separation of the metabolites was carried out in a C18 Nucleosil column (250×4 mm, particle size 5 μm; Macherey-Nagel, Germany). At a temperature of 30° C., the testosterone metabolites were analyzed and quantified by UV absorption detection at 246 nm and 285 nm (to detect dihydrotestosterone) using synthetic reference substances.

The mobile phase consisted of water (A), methanol (B) and acetonitrile (C), and HPLC measurement was conducted with different isocratic elutions: first, 60% A, 25% B and 15% C (12 min), then 45% A, 40% B and 15% C (3 min), and finally 45% A, 45% B and 10% C. Total runtime was 45 min per sample.

FIG. 5) Semiquantitative real-time RT-PCR on cardiac expression of androgen receptor in healthy, hypertrophic and LVAD-supported human hearts (A) and in non-hypertrophic and hypertrophic rat hearts (B). C shows androgen receptor expression before and after testosterone treatment (100 μM) of cultivated rat heart muscle cells. For details, please refer to “Study Description, General” and Tables 1 A and B.

FIG. 6) Semiquantitative real-time RT-PCR on cardiac expression of different genes in healthy and hypertrophic rat hearts (SHR). For details, please refer to “Study Description, General” and Tables 1 A and B.

FIG. 7) Semiquantitative real-time RT-PCR on cardiac expression of alpha-MHCs in healthy, hypertrophic and LVAD-supported human hearts (A) and in non-hypertrophic and hypertrophic rat hearts (B). C shows alpha-MHC expression before and after testosterone treatment (100 μM) of cultivated rat heart muscle cells. Isolation of heart muscle cells from adult rats was conducted as described by Thum and Borlak (2000a). Twenty-four hours after isolation, cultivated heart cells were treated with 100 μM testosterone for 8 h, removed and centrifuged at 1200 RPM and 4° C. for 5 min. Resulting cell pellet was shock-frozen in liquid nitrogen and stored at −80° C. until further analysis.

For details on the RT-PCR studies, please refer to “Study Description, General” and Tables 1 A and B.

FIG. 8) Freshly isolated rat heart muscle cells under phase contrast microscope.

FIG. 9) Potential androgen receptor binding sites in promoter of human, rat and mouse alpha-MHC genes.

Using the GEMS Launcher Release 3.0 Software (Genomatix, Germany), a matrix for the androgen receptor binding site was developed using information on sequences obtained from the transcription factor database TRANSFAC 4.0 (http://transfac.gbf.de/TRANSFAC/) and from different publications (Karvonen et al., 1997, Claessens et al., 2001; Cleutjens et al., 1996). It was thus possible to develop a matrix for the androgen receptor binding site with highly conserved bases (5′-XGXXCGGGGAGTTCT-3′). Using this matrix, binding sites for the androgen receptor in human, rat and mouse promoters of alpha-MHC gene were identified.

FIG. 10) Semiquantitative real-time RT-PCR on cardiac expression of different genes in healthy, hypertrophic and LVAD-supported human hearts and non-hypertrophic and hypertrophic rat hearts. For details on the RT-PCR studies, please refer to “Study Description, General” and Tables 1A and B.

1-23. (canceled)

24. A method treating cardiac hypertrophy, said method comprising:
administering to a patient an agent comprising a substance that increases testosterone concentration in cardiac tissues.

25. The method of claim 1, wherein said agent comprises at least one substance from the following groups:
a) testosterone
b) substances with effects similar to those of testosterone;
c) testosterone mimetics; or
d) substances that enhance testosterone synthesis

26. A method for treating cardiac hypertrophy, said method comprising administering to a patient an agent comprising a substance that inhibits or eliminates metabolites from testosterone metabolism.

27. The method of claim 3, wherein said agent comprises the following substances that inhibit testosterone metabolism, especially synthesis of dihydrotestosterone, preferably:
a) cytochrome P450 (CYP) mono-oxygenase inhibitor; and/or
b) steroid reductase inhibitor; and/or
c) isomerase inhibitor; and/or
d) 17-beta-hydroxy-steroid-dehydrogenase inhibitor; and/or
e) 3-beta-hydroxy-steroid-dehydrogenasen inhibitor; and/or
f) aromatase inhibitor.
